(19) United States
(12) Patent Application Publication
(10) Pub. No.: US 2008/0260578 A1
(43) Pub. Date: Oct. 23, 2008

(54) Device for Treating Goods with the Aid of an Electric Discharge

(76) Inventors: Jurgen Engemann, Wuppertal (DE); Axel Schwabedissen, Wuppertal (DE)

Correspondence Address: K.F. ROSS P.C., 5683 RIVERDALE AVENUE, SUITE 203 BOX 900, BRONX, NY, 10471-0900, US

(21) Appl. No.: 11/663,926

(22) PCT Filed: Sep. 20, 2005
(86) PCT No.: PCT/DE2005/001651

§ 371 Date: Jan. 4, 2008

(30) Foreign Application Priority Data
Oct. 12, 2004 (DE) 10 2004 049 473.4

(51) Int. Cl. G01N 21/77; B01J 19/08

(52) U.S. Cl. 422/56; 422/186.04; 422/186.12

(57) ABSTRACT

The invention relates to a device (10) for treating goods (14) with the aid of an electric discharge in a receiving chamber (13) for the goods, which is defined by a wall (11, 11a, 11b, 12a, 12b, 12c, 12d, 12e) made of a dielectric material having at least two electrodes (16a, 16b) arranged on the outer side thereof (15). The invention is characterised in that at least one counter-electrode (21) is coupled in a capacitive manner to both of the outer electrodes on the inner side (20) of the wall (11).

The invention relates to an apparatus for treating objects with the help of an electric discharge according to the preamble of claim 1.

The treatment of objects within the meaning of the present patent application shall be interpreted in particular as the preservation, disinfection or sterilization of various objects. The objects may be, for example, foodstuffs such as vegetables or fruits, but also cosmetics, medical devices or the like.

The treatment of objects as defined by the present patent application, however, also includes other treatment processes during which the objects are bleached or oxidized, for example. Finally, the term “treatment” also refers in general to the surface 1 modification of the objects.

The invention relates in particular, but not exclusively, to an apparatus where, with the help of an electric discharge generated in the holding chamber, ozone or UV radiation is produced for the purpose of a partial or complete sterilization of objects.

This type of treatment is used particularly for devices and commodities from the medical and pharmaceutical sectors, but also for cosmetics and foodstuffs.

Medical commodities and consumables must be sterilized in the majority of cases. This shall be understood as the destruction of all living microorganisms as well as their dormant stages (spores). For pharmaceutical packaging, cosmetics and foodstuffs such as fruit or spices, frequently a reduction in bacteria (disinfection/preservation) is sufficient. A whole series of methods are available for sterilization and/or disinfection purposes, which are applied without the help of an electric discharge and the use of which depends on the material and geometry of the objects to be sterilized. The classic method is the treatment with superheated steam (autoclaving, T>121° C.). Due to the increased use of heat-susceptible materials, such as polymer-based plastics, there is a need for “cold” sterilization and disinfection methods operating at low temperatures. This also applies for the reduction of the bacterial count in foodstuffs and cosmetics, which must also be treated at no higher than 50° C.

Important low-temperature processes aimed at the complete or partial sterilization include ethylene oxide (EO) sterilization, sterilization by means of radioactive radiation (β, γ rays) or by means of UV radiation. In addition, gas-plasma sterilization, for example according to U.S. Pat. No. 4,643,876 or according to EP 0 278 623 B1 on the basis of H₂O₂ or on the basis of peracetic acid, for example according to U.S. Pat. No. 5,084,239 or according to EP 0 387 022 B1 has become increasingly important, the sterilizing effect being substantially due to the H₂O₂ steam that oxidizes the spore casing of the microorganisms. A different, very strong oxidation agent, which can be employed at room temperature, is ozone (O₃). It has been used for quite some time in aqueous solutions for the sterilization of drinking water, for the nonchlorine bleaching of paper or as a gas for reducing bacteria in foodstuffs of all kinds as well as for cleaning and neutralizing odors in the ambient air. Ozone is a cost-efficient and environmentally friendly alternative to other chemical oxidation agents since it can be produced from the oxygen contained in the air and produces no toxic residue, but instead decomposes back into oxygen after the treatment. Also gaseous ozone can be used for the sterilization of medical products, if the concentration is sufficiently high and at the same time the humidity is relatively high (>85%) (see for example Ishizaki et al, Inactivation of the Silas Spores by Ozone, J. Appl. Bacteriol. 60, pp. 67-72 (1986)).

In the case of the majority of the known apparatuses used for sterilization by means of ozone, the ozone is not produced in the actual treatment chamber, but in a spatially separated ozone generator, and is then supplied to the treatment chamber via pipes and valves. Low-pressure methods, for example according to U.S. Pat. No. 3,719,017 or according to WO 2003/039607 are known that are still in part undergoing clinical testing, as well as methods employed at atmospheric pressure, for example according to U.S. Pat. No. 5,868,999. The production of ozone is typically carried out by operating a “dielectrically impaired discharge” excited with high voltage in an oxygen-containing gas. This type of gas discharge is also referred to as a barrier discharge due to the electrical insulation provided on the electrodes. The barrier discharge cleaves the oxygen molecules in the chemically very active atomic oxygen, which immediately bonds with the closest oxygen molecule to form ozone (O₃). This reaction is very quick and exothermic. Ozone is not stable and decomposes under the influence of heat and catalysts (contact with vessel walls and/or the sterile product). The heat generated by the flow of current between the electrodes and the chemical reaction therefore contributes directly to the decomposition of the ozone, for which reason a variety of apparatuses used for the sterilization by means of ozone are specially equipped to cool the ozone that is produced and/or the discharge electrodes, see for example U.S. Pat. No. 5,002,738 and U.S. Pat. No. 5,169,606.

The above-mentioned low-temperature sterilization and disinfection methods are associated with the following disadvantages:
The sterilization by means of β and γ rays is relatively expensive and demands strict safety measures. The treatment of foodstuffs using these methods is not permitted in Germany and other countries.
Due to the shadow effect, UV radiation cannot be used for the treatment of products with complicated, three-dimensional geometries.
The sterilization by means of EO in a pressure chamber at 4.5 bar is essentially associated with three disadvantages:
   1. Pure EO is flammable, which is why it is mixed with chlorofluorocarbons (CFCs) (12% EO, 88% CFC). Due to their damaging effect to the ozone layer, CFCs are largely banned, so that alternatives are required.
   2. EO is toxic and carcinogenic.
   3. Due to the toxic properties of EO, the sterile product must be rinsed with air for about 12-15 hours after treatment. This prevents the quick sterilization of larger quantities of medical products.
In the case of gas-plasma sterilization or ozone water vapor sterilization, the products must be placed in a special vacuum chamber since the process is carried out in a pressure range of about 0.2 to 20 mbar. This requires expensive vacuum technology (pumps, valves, pressure sensors; etc.), along with the supply of the effective agent in sufficient concentration (evaporator and/or sublimator, see for example U.S. Pat. No. 5,876,666 or U.S. Pat. No. 5,904,897). Furthermore, a heated chamber is required when using H₂O₂ or peracetic acid to guarantee sufficient steam pressure or cooling of the electrodes is required with ozone sterilizers to suppress the decomposition of the ozone. In the case of plasma sterilization also the excitation of the plasma typically occurs at a frequency of 13.56 MHz and 2.45 GHz, meaning relatively expensive high frequency generators and tuning networks must be integrated in the systems.

In general, it should also be noted that in the case of those methods using a gaseous effective agent, this agent is generally fed many times through the entire treatment chamber. The sterilization of medical objects and products is generally carried out in packaging to prevent cross-over microbial growth after treatment. This packaging is therefore semi-transparent, meaning provided with pores that allow the active agent to penetrate but keep the bacterial spores and microorganisms out. A suitable material is Tyvek® (DuPont Inc.), for example. Thus, the packaging represents another obstacle for the active agent and premature chemical decomposition or steam condensation of the active agent may occur on the outside the packaging. The feeding of active agent from the generator into the treatment chamber is frequently associated with a further loss of concentration of the active agent on the surface of the feed pipes and/or valves.

The invention is based on an apparatus according to WO 2003/059400 A1. There an apparatus for the treatment of objects, namely for the sterilization of products, is described, where an electric discharge is used to generate ozone for the sterilization of the objects. According to FIG. 1 of this published prior art, a disinfection container is provided on whose outside two flat electrodes are provided. On the inner face of the container, an electrode structure with corresponding flat electrodes that are parallel to the outer electrodes is provided.

Proceeding from the state of the art, it is the object of the invention to further develop an apparatus for treating objects according to the preamble of claim 1 such that a reliably predetermined operation becomes possible, while being easy to produce.

The invention achieves this object with the characteristics of claim 1, particularly with those of the characterizing part, and accordingly is characterized in that at least one counter-electrode that is coupled in a capacitive manner to the two outer electrodes is fixed on the inner face of the wall.

The principle of the invention is substantially to mount two electrodes on the outer face of the wall of the holding chamber and one electrode inner face the wall. The inner electrode is coupled in a capacitive manner to the two outer electrodes. This means that an alternating current can be applied to the two outer electrodes and that a voltage is only induced on the inner, capacitively coupled counter-electrode from the outer electrodes. The inner electrode is insulated, meaning provided separately from the outer electrodes and separately from a power supply unit. In particular, no electrical feed cables are connected to the inner electrode. Apertures in the wall of the holding chamber for guiding through electrical feed cables can therefore be dispensed with.

Due to the fact that the inner electrode is fixed to the inner face of the wall, very precise positioning of the three electrodes relative to each other is possible. The predefined thickness of the wall also precisely defines the distance of the outer electrodes from the inner electrode. This makes it possible to produce a surface discharge that can be predetermined with great precision. This surface discharge may fire at an edge of the inner electrode, meaning directly inside the holding chamber, so that the electric discharge fires directly in the holding chamber for treating the objects. The UV radiation and/or the ozone to be produced for sterilization or disinfection due to the electric discharge can therefore act directly on the objects provided in the holding chamber.

The apparatus according to the invention produces a surface discharge as the electric discharge, which is a special technological variation of the barrier discharge and referred to as surface barrier discharge. The surface discharge was mentioned by S. Masuda for the first time (S. Masuda et al, IEEE Trans. Ind. Appl. 24, 223-231 (1988), U.S. Pat. No. 4,666,679). Unlike a volume-barrier discharge, this discharge does not fire across a gap between electrodes mounted parallel to each other, but on the surface and at the edge to the insulation of one of the electrodes. This type of barrier discharge is characterized by extremely high efficiency of ozone production.

The electric discharge is therefore configured as a surface discharge for the apparatus according to the invention and therefore clearly differs from the electric discharge described in the illustrated embodiment according to FIG. 1 of WO 2003/059400 A1. In that patent, a volume discharge is clearly used due to the required air gap between the two inner electrodes and the inner face of the wall of the container. It is essential for such volume discharges that the distances between the electrodes be maintained, with great precision since the distances between the electrodes are crucial for the electric discharge to be produced. In the state of the art on which the invention is based, positioning of the inner electrode structure with the required degree of precision is only possible with a highly complex configuration.

According to the apparatus from WO 2003/059400 A1, the ozone is produced right in the closed container in which the objects to be treated are held. However, due to the type of barrier discharge (volume discharge) used, a container with rigid walls is required: To ensure even discharge, the inner and outer electrodes must be oriented parallel to each other and rigidly fixed in place.

This, however, is practically impossible, particularly along the entire electrode surface, since even small changes of the distance between the electrode surfaces may greatly affect the type of electric discharge.

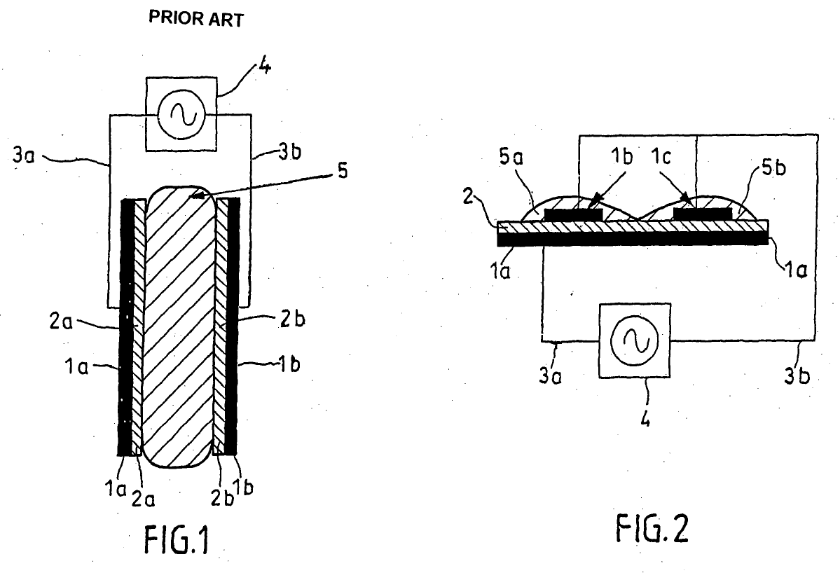

In the apparatus for sterilizing objects by means of ozone described in EP 0863772 B1 also no electrode fixed on the inner face of the wall of the holding chamber is provided. Here as well, at least a minor air gap develops between the inner electrode that is mounted loosely in the plastic container and the dielectric wall of the lower electrode according to FIG. 2 shown there, on which electrode the plastic bag rests. Depending on how the inner electrode with the plastic bag is positioned between the two plate electrodes fixed in the treatment chamber, different electric discharges are produced.

When used for sterilization and disinfection purposes, the apparatus according to the invention enables treatment of the objects at relatively low cost and without the need to adhere to special safety measures. Treatment of foodstuffs, for example, is permitted with the apparatus according to the invention.

Due to the fact that the ozone and UV radiation are produced directly in the holding chamber when the apparatus is used for sterilization or disinfection purposes, the apparatus according to the invention also enables the treatment of products having more complicated, three-dimensional structures.

The apparatus according to the invention can provide a sufficiently large amount of ozone and UV radiation over a sufficiently long period of time within a closed package, so that a sterilization of a temperature-susceptible, medical product or a disinfection or partial disinfection of cosmetics or foodstuffs provided inside the packaging can be performed. It is preferable if the treatment is carried out at atmospheric pressure inside the packaging, thus eliminating a costly vacuum solution. Unlike WO 2003/059400 A1, the wall of the holding chamber may also flexible, allowing a variety of materials to be considered for the wall of the holding chamber and also allowing the use of conventional packaging materials for the objects. This also enables use of the apparatus according to the invention for transporting or storing the objects.

The active agent, meaning for example the ozone combined with UV radiation, is produced by a surface discharge, for example in atmospheric air inside the packaging of the product to be treated, meaning inside the holding chamber. For this purpose, at least two metallic electrodes are provided on the outside the packaging, which is made, for example, of plastic such as polyethylene (PE) or another polymer (PA, PVC, PET, . . . ), the electrodes being mounted such that no air gap is present between metal and dielectric. In the simplest case, this may be implemented by applying a thin metal film by gluing or also by imprinting (screen printing etc.) or vapor deposition using PVD (=physical vapor deposition) methods. The electrodes provided on the outer face of the packaging may be connected electrically to a power supply, with alternating current being applied. The amplitude of the voltage is preferably several kV to a maximum of 20 kV, the frequency is preferably 1 to 30 kHz. The distance of the outside electrodes from each other is selected such that no electrical breakdown can occur in the outside air when the maximum voltage is applied. Alternatively or additionally, also an insulating section or barrier may be provided between the two outer electrodes to prevent electrical breakdown.

On the inner face of the wall, opposite the outer electrodes, a metallic structure is applied as the counter-electrode, for example also made of copper or aluminum film, the edges of which have slightly smaller dimensions than those of the outer excitation electrodes. The inner electrode is a counter-electrode coupled in a capacitive manner. This electrode “floats” electrically, meaning the counter-electrode has no metallic connecting line to the outside the packaging. Just like the outer electrodes, it is applied directly to the inner face of the wall (glued on, imprinted or applied by means of PVD methods). As soon as the ignition field strength has been reached, plasma develops in the form of a transition zone along the edge of the inner electrode facing the insulation. This plasma produces free electrons, ions, radicals (for example atomic oxygen) as well as UV radiation through the recombination of electronically excited molecular and atomic species, particularly ozone from the ambient oxygen in the holding chamber. The amount of ozone that is produced is proportional to the length of the transition zone. Therefore it is beneficial if the counter-electrode coupled in a capacitive manner has the longest possible edge.

The plasma-generation process is carried out until the desired degree of sterilization has been reached. This depends on the geometry and surface properties of the product as well as on the field of application.

To prevent the flexible packaging, for example, from covering parts of the surface of the objects to be treated and impairing sterilization, a slight excess pressure can be produced in the holding chamber by blowing in air or another oxygen-containing gas mixture immediately before closing, for example welding, the wall. Furthermore, the efficiency of the ozone treatment process can be improved in that the moisture level inside the packaging is raised by spraying the packaging with a fine mist of water or water droplets prior to welding its seams.

The apparatus according to the invention enables the production of an electric discharge in the form of a surface discharge, which is clearly modified from the known surface discharges according to Masuda. The new electrode geometry enables particularly efficient ozone production, with a particularly high ozone concentration inside the holding chamber. One advantage of this method is that the active agent (such as ozone and UV radiation) is produced where it is required, meaning inside the packaging. Furthermore, no complex vacuum technology is required since the process can take place at atmospheric pressure (cost and time savings).

The active agent is produced only inside the packaging and will automatically decompose after turning off the power supply. The half-life value for the decomposition of ozone is about 20 minutes, which means that long degassing or ventilation and evacuation periods are eliminated. No toxic residue remains since O₃ decomposes into oxygen or oxidized decomposition products of organic substances, meaning essentially CO₂.

A further advantage is that no effort is required to produce an active agent and feed it to the treatment space. Due to these cost savings, the method is also suited for products with little value.

A further advantage is that a power supply for the necessary frequency range at which the discharge can be operated (typically several kHz) can be produced at considerably lower cost than a high-frequency generator with tuning network.

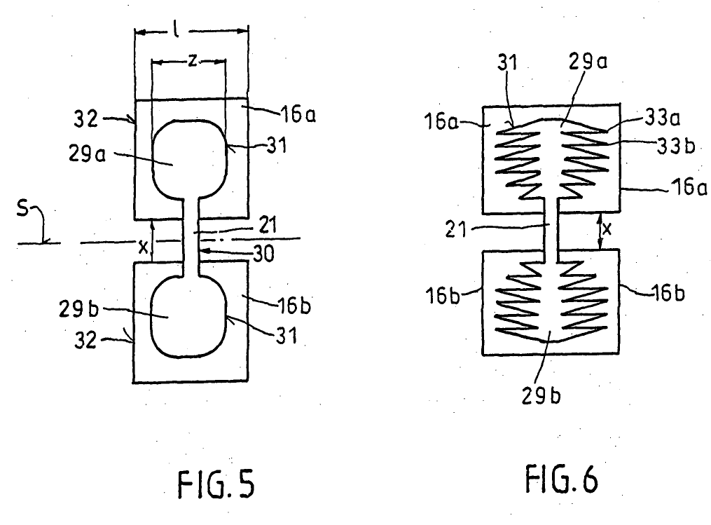

A further advantage of the invention is that particularly flexible single-use packaging can be used. Unlike the rigid containers with rigid electrodes and special power-cable routing for the inner electrodes disclosed, for example, in the illustrated embodiments according to FIGS. 5 and 8 of WO 2003/059400 A1, cost-efficient, recyclable packaging made of PE film or similar material with adhesive or imprinted electrodes can be used for the apparatus according to the invention. The electrodes can in particular be part of labels that are glued directly on the packaging. Both the inner electrode and the outer electrodes can be configured as part of a label.

It should be noted that the treatment of objects primarily described in the present patent application emphasizes sterilization or a reduction in bacteria, disinfection or the like. However, a number of additional treatment methods, particularly surface modifications of varying types, can be considered, in which an electric discharge plays a role. The wording of the present patent application, according to which the treating of objects is carried out with the help of an electric discharge, means in particular that the electric discharge generates secondary products or effects, such as ozone or UV radiation, for example. The wording according to which the treatment of the objects is carried out with the help of an electric discharge, however, also includes such apparatuses and treatments in which the treatment of the objects, particularly the surfaces thereof, is carried out directly by the electric discharge, meaning a plasma.

It should be noted that in the event the electric discharge is intended to produce ozone or UV radiation, a separate agent does not need to be fed into the holding chamber, since in the majority of packing situations for diverse objects air is already present in the holding chamber. The apparatus according to the invention therefore does not require separate input of an agent.

In different applications, the feeding of an agent into the holding chamber, for example in the form of a gas mixture or a gas, may certainly be desirable and expedient.

The apparatus according to the invention can be connected to a power supply. This means that the power supply is not actually part of the apparatus, but may be provided, for example, in a fixed location. Different devices be designed as transport receptacles for objects, for example, can be connected to the power supply for producing an electric discharge and for treating the objects and then may be separated, meaning disconnected, from the power supply after the treatment has been carried out.

According to an advantageous embodiment of the invention, the apparatus is configured as a transport container and/or as a storage container for the objects. This way it is possible to configure a conventional, meaning typical, transport container or storage container—with the exception of the provision of the electrodes—for objects, such as foodstuffs, as the treatment apparatus for these objects. In the simplest case, the two outer electrodes and the inner counter-electrode are fastened to conventional food packaging, the wall defining the holding chamber being made of plastic, for example. The objects can then be packaged in the conventional manner, the transport container, in other words the packaging, being closed in the conventional manner, so that the objects located in the holding chamber, meaning inside the transport package, are accommodated with permanent protection. After packing the objects, meaning when these are enclosed inside the transport container, an electric discharge can be produced inside the holding chamber by connecting the outer electrodes and a power supply to generate ozone as well as UV radiation to carry out a disinfection or sterilization process. After carrying out the treatment, the objects provided in the holding chamber can be protected particularly well when the wall is made to be impervious to bacteria and/or gas.

Making the apparatus according to the invention as a transport container or as a storage container for the objects enables very inexpensive manufacture of such a container, compared to conventional transport devices or storage devices only the additional electrodes must be provided. However, since these, as described above, can be applied for example as metal film, very little additional costs are incurred compared to conventional transport and storage packaging systems.

Furthermore, this configuration of the invention is associated with advantages in that separate storage devices or separate treatment spaces or chambers for the objects can be eliminated. The objects can be stored and treated in one and the same receptacle.

In addition, this configuration of the invention offers the advantage that the treatment of the objects can now also be performed at the point of use, meaning where the objects are located anyhow, thus possibly eliminating additional transportation costs. For example, the disinfection or sterilization of fruit can be carried out wherever they have just been harvested, preferably immediately after placing the fruit in the holding chamber, meaning the packaging, and after completely sealing the packaging.

The true short-cut is that the wall of the packaging forms the insulation barrier for generating a dielectrically impaired discharge.

According to a further advantageous embodiment of the invention, the wall is formed by conventional packaging for the objects, with the exception of the electrodes. The provision of the electrodes to construct an apparatus according to the invention thus requires very little additional expense.

According to a further advantageous embodiment of the embodiment, the wall is made of plastic, particularly of PE (polyethylene), PA (polyamide), PVC (polyvinylchloride), PET (polyethylene terephthalate) or the like. This offers the possibility to use conventional packing materials.

According to a further advantageous embodiment of the invention, the wall is impervious to gas. This enables a lasting, safe accommodation of the objects inside the holding chamber, without the risk of renewed bacterial growth when the treatment carried out is a sterilization process.

According to a further advantageous embodiment of the invention, the apparatus is configured as single-use packaging. This allows conventional handling of packaging systems for certain objects.

According to a further advantageous embodiment of the invention, the apparatus is configured as reusable packaging. This way the apparatus can be recycled, which is handy for certain objects and may result in lower costs.

According to a further advantageous embodiment of the invention, the two outer electrodes can be connected with the terminals of a power supply. In the simplest case, a power supply is provided with two connecting wires whose the ends are formed as terminals that can be used to establish a detachable electrical connection to the two outer electrodes. At the same time, meaning when the electrical connection is established, provision may also be made for a mechanically detachable connection.

In the event that the outer two electrodes are fixed to the outer face of the wall, and for example each have an outwardly exposed outer face, the terminals of the power supply can be brought into direct contact with the two outer electrodes, for example with the help of a magnet. Here it is particularly important that the wall defining the holding chamber can also have be made flexible.

In one alternative embodiment of the invention, the outer electrodes are provided with contact plugs that can be detachably connected directly to the terminals of the feed lines of the power supply.

Finally, it is also possible not to affix the two outer electrodes to the outer face of the wall, but instead to move the two outer electrodes, which in this case are fixed to the connecting lines of the power supply, directly against the outer face of the wall for treating the objects. This may also be expedient in some applications. It is true that also this case is associated with the problem that very precise positioning of the outer electrodes relative to the outer face of the wall is required. However, this type of positioning can be performed from the outside, which guarantees in a simple manner that no air gap is left between the electrodes and the outer face of the wall. In addition, for example markings for the outer electrodes can be provided on the outer face of the wall for positioning the electrodes. Alternatively, also markings may be provided on the outer face of the wall that indicate the precise position of the inner electrode.

It is preferable, however, if the two outer electrodes are fixed to the outer face of the wall.

According to a further advantageous embodiment of the invention, the wall comprises a closable opening into the holding chamber. Such an opening can be formed, for example, by a door mounted pivotally or slidably, for example, on a wall, which in this case is preferably rigid. Alternatively, the access opening can be formed by a slide fastener, which is particularly expedient when the wall is flexible and forms, for example, a bag-like receptacle provided with a slide fastener.

Finally, there is also the possibility of making the holding chamber a box- or cup-shaped receptacle having a detachable cover. It is also conceivable that the apparatus comprises a bottom tray, as is known for example for transporting fruit, that is closed at the top by a flexible plastic film.

It is possible to configure the access opening such that it can only be closed one time, but it can also be configured such that it can be reopened.

According to an advantageous embodiment of the invention, the wall is configured to be flexible in sections or in its entirety. If the wall is made entirely flexible, it is possible, for example, to provide a bag-like receptacle. The flexible configuration of the wall, which can be provided at least in part or in sections, offers the advantage of requiring little space for storing the container when it is not in use. In this case it can be collapsed or folded, for example. In addition, a flexible wall may accommodate different volumes inside the holding chamber, so that an increased volume of the holding chamber can be achieved, for example, by increasing the pressure inside the holding chamber. This is particularly advantageous when shadow zones need to be avoided when treating the objects, for example by means of ozone or by UV radiation, so that a treatment of the objects along their entire surface becomes possible.

Alternatively to a flexible configuration of the wall, provision may also be made for the wall to be configured substantially rigid.

This may be advantageous under certain circumstances for certain objects, for example for medical devices or apparatus, when storage containers according to the state of the art already have rigid walls for these objects. The invention also enables the container to be a blister package provided with two outer and one inner electrode.

It shall be noted that the apparatus according to the invention must comprise at least two outer electrodes and one inner electrode. Further electrodes can also be installed, for example a further inner electrode and a further pair of outer electrodes provided for this inner electrode.

According to a further advantageous embodiment of the invention, an inner face and/or an edge of the inner electrode is exposed in the holding chamber. Due to the fact that at least edges of the inner electrode are exposed, meaning not covered by a dielectric material layer, the electric discharge can be generated in the form of a surface spark. At the same time, this shape enables the electric discharge to take place unshielded in the holding chamber. The electric discharge is accordingly not shielded by a cover from the actual holding chamber, as is provided for example in the illustrated embodiment according to FIG. 1 of WO 2003/059400 A1. This increases the efficiency of the treatment.

According to a further advantageous embodiment of the invention, the inner electrode is positioned relative to the two outer electrodes and dimensioned such that with a vertical projection of the inner electrode on the outer electrodes, the outer edge of the inner electrode is located substantially entirely within the outer edge of the outer electrodes. It should be noted that preferably the inner electrode and the two outer electrodes are mounted directly opposite from each other and are only separated by the insulation wall of the holding chamber. The two outer electrodes are mounted at a distance from each other and are connected to the power supply via separate connecting lines. The inner electrode preferably comprises two heads and is configured, for example, substantially dumbbell-shaped, the two heads being electrically connected to each other via a narrow bar. The surface of the two heads is smaller than the surface of the associated outer electrode, respectively. If the inner electrode were projected vertically on the outer electrodes, the outer edge of the inner electrode would be located substantially inside the outer edge of the two outer electrodes. The wording “substantially” is intended to take into consideration that the thin connecting bar is not taken into consideration.

According to a further advantageous embodiment of the invention, the inner electrode, particularly at least one head of the inner electrode, has a shape with a plurality of areas with directional changes. This embodiment of the invention makes it possible for the inner electrode to have a particularly long edge, thus creating a particularly long transition zone for the electric discharge. This way, a large amount of ozone and UV radiation is generated.

An area with directional change is considered as the location at which the direction must be changed particularly abruptly when passing along the edge or at which a curvature of the edge changes.

It is preferable if the outer electrodes and the inner electrode are configured symmetrically relative to a common plane of symmetry.

This, however, is not absolutely required. The two outer electrodes and the inner electrode can also be configured asymmetrically.

According to a further advantageous embodiment of the invention, the pressure inside the holding chamber is greater than outside the chamber. This way, the objects can be treated along their entire surface.

According to a further advantageous embodiment of the invention, a pressure ranging between 50 hpA and 150 hpA, particularly atmospheric pressure, prevails inside the holding chamber during the discharge. This way, the apparatus can be operated without complex vacuum systems, enabling cost-efficient production and cost-efficient operation of the apparatus.

According to a further advantageous embodiment of the invention, a gas mixture with an oxygen-containing gas is present inside the holding chamber in addition to the objects to be treated. This enables the production of ozone caused by, meaning with the help of, an electric discharge.

According to a further advantageous embodiment of the invention, the water-vapor content in the holding chamber is raised, particularly by supplying water vapor. This enables improved efficiency of the ozone treatment.

According to an advantageous embodiment of the invention, the inner electrode is mounted directly on the wall, particularly without leaving an air gap. This way, on the one hand particularly easy installation of the inner electrode on the wall of the holding chamber becomes possible because the inner electrode can be, for example, glued, vapor deposited, imprinted or applied directly to the wall in another manner, without necessitating separate fastening elements. Furthermore, the direct application of the inner electrode to the wall also enables very precise positioning of the inner electrode relative to the outer electrodes because the distance of the inner electrodes to the outer electrodes is defined by the thickness of the wall. Due to the manufacturing process of the wall, for example, the wall thickness however will be known within very tight tolerances and can be predetermined. The physical parameters for the electric discharge can therefore be predetermined with great precision.

Equally and independently thereof, for the same reasons, it is advantageous when the outer electrodes are provided directly on the outer face of the wall, particularly without leaving an air gap.

According to a further advantageous embodiment of the invention, the electrodes are made of metal, in particular silver, gold, stainless steel, aluminum or copper or an alloy comprising at least one of these metals. This embodiment of the invention takes into consideration that, in the case of an apparatus configured as single-use packaging, metals that are easy to oxidize, such as copper, may be used.

According to a further advantageous embodiment of the invention, at least one electrode is formed by a metal film, which is glued to the wall.

Alternatively, at least one electrode is imprinted, vapor deposited or applied to the wall by a sputter method. This enables a particularly simple and cost-efficient production of the electrodes.

The electrodes can also be imprinted on the outer face of the wall in the form of lettering, for example.

The electrode may be configured as part of a label, which can be attached, in particular glued, to the wall, particularly a wall configured as packaging.

The label may comprise, for example, a plastic wrapping for the electrode. Alternatively, the label may be made of textile material or paper or cardboard material, or it may comprise a combination of different materials, for example also in a type of sandwich design.

The electrode may also be formed by a metal film as part of a label. In particular when the label is made of a plurality of material layers, the electrode can be applied, for example glued, vapor deposited, imprinted or sputtered, on one of the material layers.

The label can be attached to the wall by gluing or by another suitable fastening method, optionally also by thermal welding. In the event that the wall is configured as packaging, which is made of plastic film, for example, it is particularly advantageous to glue the labels comprising the electrodes on the packaging.

The label may be provided with information on the outer face, for example an identification or batch number or a bar code, which is printed for example. The label may also be associated with an indicator, for example litmus paper, that shows a treatment state of the objects, for example a sterilization level that the objects have achieved. The display apparatus may also be configured as an indicator, for example for ozone, which changes color when reaching a certain treatment state or after a chemical treatment has been carried out.

Particularly if the electrode is part of an adhesive element, such as a label, the electrodes can be produced particularly easily and inexpensively and attached to the wall. At the same time, particularly thin packaging material can be selected if the wall is configured as packaging for the objects. Since the thermal load applied to the wall is greatest when making plasma in the vicinity of the electrodes, the mean thermal load applied to the packaging in this region can be reduced by appropriately selecting the thickness of the label. Thus, the plasma can be operated for a longer period before damaging the packaging. By configuring the labels with an appropriate thickness, the wall as such can be very thin, for example a packaging film having a wall thickness of 50 mm, without running the risk of applying a destructive thermal load during generation of plasma.

A particularly advantageous, secure and easy attachment of the electrodes to the wall can be achieved in that the electrodes or the labels comprising the electrodes are applied by an apparatus in one operation to both sides of the wall, particularly to both sides of a packaging film.

The apparatus for attaching the electrodes can be equipped, for example, with two displaceable tools, between which the wall is positioned. The tools may comprise the electrodes or the labels to be attached at their free ends that can be displaced toward each other. The tools are guided toward the two sides of the wall until the electrodes or the labels come into contact with the wall. If the electrodes or labels are adhesively attached to the wall, the electrodes or labels are moved toward the wall with their adhesive sides first.

The tools can press the electrodes or labels directly on the wall surfaces with the necessary pressing force. An embossing operation is also conceivable, optionally with, the help of thermal effects.

By applying the electrodes or labels to the wall in one, operation by a single apparatus, it is easier to position the electrodes relative to each other on the wall. The relative position of the electrodes to each other can be achieved by such an apparatus in a particularly simple manner and can be predetermined with great accuracy.

It is particularly advantageous when all electrodes, meaning the inner and the outer electrodes, are configured as parts of labels. The two outer electrodes can be carried by a common label.

According to a further advantageous embodiment of the invention, at least one electrode is made of an electrically conductive and optically transparent, meaning translucent, material. For this application, for example, indium tin oxide (ITO) or a comparable material, preferably a metal oxide, can be used. An electrically conductive and optically transparent material makes it possible to provide outer and inner electrodes also in the case of transparent or clear containers, such as glass bottles or transparent plastic films, without these electrodes being visually conspicuous in any way or even considered as unattractive.

According to a further advantageous embodiment of the invention, an alternating current ranging between 0.5 kV and 20 kV having a frequency between 100 Hz and 10 MHz, preferably between 1 KHz and 30 KHz, is applied between the two outer electrodes. This enables particularly efficient plasma production and only requires an inexpensive power supply.

According to a further advantageous embodiment of the invention, the discharge is a surface barrier discharge. This enables particularly efficient ozone generation and consequently particularly efficient treatment of the objects.

According to a further advantageous embodiment of the invention, a display apparatus is provided inside the holding chamber, which apparatus shows a treatment state of the objects.

The display apparatus can be provided, for example, by litmus paper, which displays a sterilization level reached by the objects, for example by displaying a current pH value or the like. It is also possible to display other treatment states. It is advantageous that the display apparatus is provided inside the holding chamber, which is preferably completely closed. Direct access to the objects located inside the holding chamber for the purpose of determining the treatment state can therefore be eliminated so that the holding chamber, for example in the form of single-use packaging, does not need to be opened to determine whether the desired sterilization level has been reached.

Further advantages of the invention will be apparent from the uncited dependent claims as well as the description provided hereinafter of several illustrated embodiments shown in the drawings, wherein:

FIG. 1 shows a purely schematic illustration of the operating principle of a volume discharge for an apparatus according to the state of the art,

FIG. 2 shows in an equally schematic cross-sectional illustration the operating principle of a surface discharge,

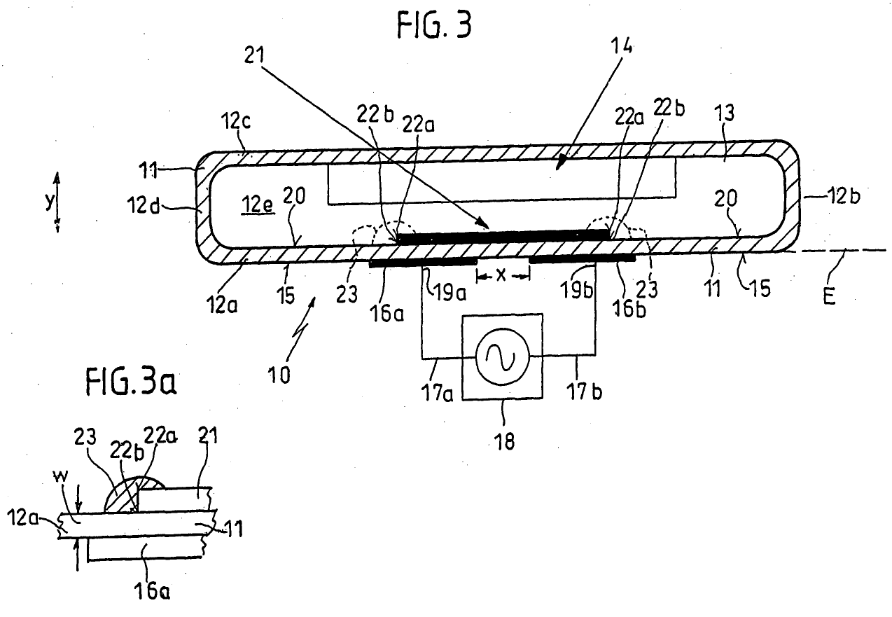

FIG. 3 is a schematic illustration of a first embodiment of the apparatus according to the invention,

FIG. 3a shows a schematic, enlarged sectional illustration of FIG. 3,

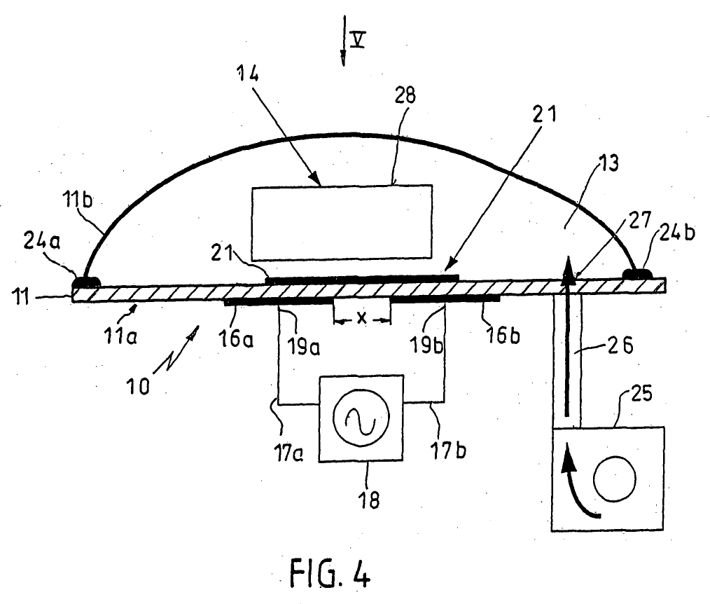

FIG. 4 is an illustration similar to that according to FIG. 3 of a second embodiment of the apparatus according to the invention,

FIG. 5 is a projected view according to the arrow V from FIG. 4 of the electrode arrangement of the apparatus according to FIG. 4, and

FIG. 6 is an illustration similar to that according to FIG. 5 of a second embodiment of the electrode arrangement according to the invention.

The apparatus according to the invention has been denoted overall with reference numeral 10 based on the illustrated embodiments in FIGS. 3 and 4. In this context, it should be noted that identical or comparable parts or elements in the different figures have been denoted with the same reference numerals for clarity and simplicity reasons, in part while adding lower-case letters.

Before describing the apparatus according to the invention, first the operating principle of a volume discharge shall be described with reference to FIG. 1:

FIG. 1 shows a first electrode 1a, configured as a flat electrode. Opposite this electrode, a substantially equally large second electrode 1b is provided, thus creating an arrangement like a plate capacitor. Each electrode is associated with a respective insulation barrier 2a and 2b provided between the two electrodes 1a and 1b. The insulation barrier 2a made of a layer or plate of dielectric material is associated with the first electrode 1a, and the second barrier 2b is associated with the second electrode 1b and attached thereto. The two electrodes are connected to a power supply 4 via connecting lines 3a and 3b. This unit produces an alternating current, so that the two electrodes 1a and 1b are at different potentials.

If the frequency and voltage are selected appropriately, an electric discharge in the form of plasma 5 is created between the insulation barriers 2a and 2b. This plasma, in a simplified analysis, forms in the regions between the two electrodes 1a and 1b in which they have the shortest distance from each other. The plasma 5 fills a volume, so that this type of electric discharge is also referred to as electric volume discharge. The important factor here is that the two electrodes 1a and 1b are positioned with great precision relative to each other, since any deviation from the target position results in different distances and consequently different formations of the plasma 5.

With reference to FIG. 2, now the principle of surface discharge will be explained in general terms, which principle is used for the apparatus according to the invention. Again, a power supply 4 is provided that applies alternating current to a first electrode 1a and a second pair of electrodes 1b and 1c via connecting lines 3a and 3b. The electrodes 1b and 1c are therefore at the same potential, while the electrode 1a has a different potential.

An insulation barrier 2 is provided between the electrode 1a and the two electrodes 1b and 1c. It should be noted that the two electrodes 1b and 1c are set at a spacing from each other.

With a suitable selection of the frequency and voltage, a transition zone, namely an electric discharge in the form of surface plasma 5a or 5b, develops substantially along the edges of the electrodes 1b and 1c. Since the discharge occurs substantially along the edges of the electrodes 1b and 1c, this is not referred to as volume plasma, but instead as a surface discharge.

The apparatus 10 according to the invention will now be explained with reference to FIG. 3:

According to FIG. 3, the apparatus 10 is shown in schematic cross-sectional view and comprises a wall 11 with a floor 12a, a ceiling 12c, a left side 12d and a right side 12b. The wall 11 borders and defines a holding chamber 13 for objects with the wall parts 12a, 12b, 12c and 12d thereof. FIG. 3 indicates by way of example rectangular objects 14 that sit against to the ceiling 12c. The objects 14 shown are in this case attached to the ceiling 12c. In the event the objects 14 are placed loosely in the holding chamber 14, the objects 14 may also rest on the ceiling 12c of the wall 11 while taking the force of gravitation into consideration. In this case, FIG. 3 is upside down, however this is irrelevant for the analysis and explanations to follow.

In addition, it should be noted that the holding chamber 13 is closed. Accordingly, a front wall section, which is not shown, is part of the wall 11. The rear wall section of the wall 11 is indicated in FIG. 3 and denoted with numeral 12e.

The wall 11 can be made of plastic or any other dielectric material. It is preferable if the wall 11 has a constant wall thickness w along its entire circumference. However, this is not required.

The wall 11 can be made of a flexible material, or it can be made relatively rigid. This will depend on the application of the apparatus 10.

In the embodiment according to FIG. 3, it will be assumed for simplicity reasons that the wall 11 is made of a relatively rigid material.

FIG. 3 shows that a first outer electrode 16a and a second outer electrode 16b are provided and attached to an outer face 15 of the wall 11. It should be noted that no air gap exists between the two electrodes 16a and 16b and the outer face 15 of the wall 11, but that the two electrodes 16a and 16b are attached directly to the outer face 15 of the wall 11.

The two electrodes 16a and 16b are supplied with alternating current having a suitable frequency and a suitable voltage via respective connecting lines 17a and 17b of a power supply 18. The free ends 19a and 19b of the respective power supply lines 17a and 17b can be detachably electrically connected to the corresponding electrode 16a and 16b.

An inner electrode 21 is mounted on an inner face 20 of the wall 11 opposite the outer face 15. The inner electrode 21 is attached directly to the inner face 20 of the wall 11 without an air gap between the electrode 21 and the wall 11.

Since the ends 19a and 19b of the associated electrodes 16a and 16b are detachable, the wall 11 with the electrodes 16a and 16b and 21 attached thereto and the objects (for example 14) inside the holding chamber 13 form a manageable assembly. The apparatus 10 is therefore suitable for use as a transport or storage container for the objects 14. If required and if the objects 14 located inside the holding chamber 13 are supposed to be treated, the apparatus can be connected to the connecting lines 17a and 17b of a power supply 18.

When the apparatus is in operation, an electric discharge develops along the edges 22a and 22b of the inner electrode 21, and specifically a surface discharge, meaning a plasma in the form of a transition zone. The developing transition zone is indicated in a dotted fashion in FIG. 3 and has been assigned reference 23. For clarity reasons, FIG. 3a, which shows an enlarged sectional view of the left edge region of the inner electrode 21 from FIG. 3 and the outer electrode 16a, illustrates the transition zone 23 schematically with a hatched area. The plasma that is produced in a kind of transition zone along the edges is filamentary plasma, meaning no APG plasma and therefore no glow plasma. By producing an electric discharge 23 inside the holding chamber 13, ozone and UV radiation are generated. Ozone and UV radiation are referred to as the agent hereinafter. This agent may interact with the objects 14 located in the holding chamber 13 and may disinfect, sterilize or otherwise treat these objects. Other types of treatment, depending on the objects at hand and depending on the gas or gas mixtures present inside the holding chamber 13, are possible. For example, also bleaching, oxidation or another type of surface modification of the objects 14 located in the holding chamber 13 is conceivable.

It should be noted that the inner electrode 21 is a counter-electrode to the two outer electrodes 16a and 16b, which counter-electrode is coupled in a capacitive manner. The inner electrode 21 is consequently not connected to any power supply lines of a power supply outside the holding chamber 13. The inner electrode 21 is completely independent from this unit. In it, voltage is induced exclusively by the outer electrodes 16a and 16b.

Due to the fact that the inner electrode 21 and the two outer electrodes 16a and 16b are attached directly to the wall 11, the position of the electrodes 16a, 16b and 21 relative to each other is accurately predetermined. Particularly the distance of the electrodes from each other to be defined in the direction of the double arrow y according to FIG. 3 is precisely defined in this region due to the thickness w (FIG. 3a) of the wall 11. As a result, the surface discharge 23 can also be predetermined with great precision.

It should be noted that it is possible to adapt the thickness w of the wall 11 of the apparatus 10 to the requirements of the desired surface discharge. On the other hand, by varying the electrode geometry, an existing and unchangeable thickness w of the chamber 11 can be taken into consideration. The two outer electrodes 16a and 16b are mounted at a distance x from each other along the plane E. The distance x is selected such that a breakdown between the electrodes 16a and 16b is prevented. If necessary, an insulator, which is not shown in FIG. 3, may also be provided between the two electrodes 16a and 16b.

FIG. 4 shows a second embodiment of the apparatus 10 according to the invention, where the wall 11 of the apparatus consists of a first wall section 11a and a second wall section 11b. The first wall section 11a may be configured relatively stiff or rigid, for example, and form a kind of support plate. The second wall section 11b may be formed by more flexible packaging, for example a film. The two wall sections 11a and 11b may be firmly connected, for example welded, to each other in the region of connecting areas 24a and 24b, thus providing a completely closed holding chamber 13 for objects 14. In FIG. 4, the rectangular objects 14 are again only indicated very schematically.

The arrangement of the electrodes 16a, 16b and 21 is comparable to the electrode arrangement according to FIG. 3, so that their description will be foregone at this point. The special feature in the illustrated embodiment according to FIG. 4 is that the apparatus 10 is associated with a blower 25 that forces air into the holding chamber 13 via a conduit 26 and an aperture 27 in the supporting plate 11a. The aperture 27 can be closed like a valve, so that the apparatus 10 can become completely gas tight or gas impermeable after treating the objects 14.

The blown-in air lifts the flexible wall 11b off the top 28 of the objects 14, allowing the objects 14 to be surrounded by air on all sides. This promotes a uniform treatment of the surfaces of the objects 14.

It should be noted that FIG. 4 should be interpreted only as a schematic illustration, because due to gravity the objects 14 rest with their material regions nearly automatically on the inner faces of the wall section 11a. Additionally, it should be mentioned that the apparatus can be moved during treatment, for example by means of vibration or shaking, thus ensuring that different areas of the objects 14 rest against the inner faces of the wall 11, which also enables homogeneous or more homogeneous treatment of the objects.

As an alternative to blowing gas in the chamber according to the embodiment according to FIG. 4 and producing excess pressure in the holding chamber 13, the same effect can be achieved by producing a vacuum outside the wall 11.

It should finally also be noted that special gases or gas mixtures, in part also further agents, can be introduced into the holding chamber 13 via the blower 25, the conduit 26 and the aperture 27. This is particularly significant for surface modifications. In the apparatus 10 according to FIG. 4, the ends 19a and 19b of the connecting lines 17a and 17b may also be detached from the outer electrodes 16a and 16b, so that the apparatus 10 can be detachably connected to the power supply 18 and forms a manageable unit that can serve as a transport container or holder for the objects 14.

The geometry of the electrodes will now be explained with reference to FIGS. 5 and 6:

FIGS. 5 and 6 each show projection substantially along the arrow V according to FIG. 4, only the inner electrode 21 and the two outer electrodes 16a and 16b being illustrated. In other words, the wall 11b, the objects 14 and the wall section 11a have been omitted for clarity reasons.

FIG. 5 shows that the inner electrode 21 is substantially dumbbell-shaped and comprises a first head 29a and a second head, 29b connected to each other via a narrow connecting bar 30. The two outer electrodes 16a and 16b have a substantially square shape and are mounted at a distance x from each other. Both the inner electrode 21 and the two outer electrodes 16a and 16b are mounted symmetrically along a plane of symmetry S.

FIG. 5 illustrates that each of the outer electrodes 16a and 16b represents a square with an edge length l. Each head 29a and 29b of the inner electrode 21 is shaped substantially as a square with rounded corners, with the square having an edge length z smaller than l, so that the projected surface of each head 29a and 29b lies within outer edges 32 of an outer electrode 16a and 16b. The outer edge of the inner electrode 21 is designated by numeral 31, the outer edge of the outer electrode 16a and 16b by numeral 32.

Since the outer edge 31 of the inner electrode 21 rests completely within the outer edge 32 of the outer electrodes 16a and 16b, with the exception of the region of the connecting bar 30, a transition zone 23 (FIG. 3) develops inside the holding chamber 13. The relevant point is that the length of the transition zone is substantially proportional to the length of the corresponding edge 31, 22a and 22b of the inner electrode 21. FIG. 5 illustrates one edge 31 based on the dumbbell shape of the inner electrode 21, however this edge is not very long. A further embodiment of the inner electrode 21 according to FIG. 6 shows an inner electrode 21 sort of like a double pine tree or fern shape, the edge or contour 31 of the inner electrode 21 being provided with a plurality of areas with switchbacks 33a and 33b. These are areas at which the direction changes when passing along the edge 31, meaning that for example the curvature changes from a right-hand curve into a left-hand curve, or areas, at which the direction changes abruptly, sort of like a discontinuous area, in particular in a zigzag shape. Consequently, particularly long edges 31 are provided on the inner electrode 21 according to FIG. 6, which edges guarantee the production of a particularly long transition zone 23 and thus the generation of a large amount of ozone or UV radiation.

It is particularly relevant that the electrodes can also be configured serrated or alternatively serpentine-shaped. The structuring of the electrode contour is only important in the plane E.

Inner electrodes 21, which are not shown, may additionally comprise a series of openings. For example, each head 29a and 29b of an electrode 21 according to FIG. 5 may be provided with a plurality of holes, thus significantly increasing the edge length of the edge regions. This way, ozone generation can be improved further.

The electrode dimensions can be in the millimeter or centimeter range. The smaller the overall dimensions of the apparatus 10, the smaller also the electrode surfaces can be selected.

The illustrated embodiments according to FIGS. 3 and 4 relate both to apparatuses 10 with flexible walls and to apparatuses with substantially rigid walls. In the case of a flexible apparatus, it is also conceivable, for example, to form the wall from a flexible plastic sack or bag that is formed with an access opening to the holding chamber 13 by means of a slide fastener. This way, it is possible, for example, to place medical devices in a physician's practice in the holding chamber 13 without difficulty and to close the holding chamber with the slide fastener. After this, the medical devices can be treated, for example disinfected.

Alternatively, the wall 11 can also be made of relatively rigid material, an access opening for the holding chamber being formed by a door, a flap, a cover or the like.

Finally, also those apparatuses are possible, in which the chamber is permanently closed tightly and completely by the wall 11, as is indicated for example in FIG. 4, showing welds 24a and 24b. In this case, for example, conventional transport packaging for foodstuffs can be closed with a vacuum sealing machine or the like. The apparatus according to the invention then enables treatment of the completely packaged objects, allowing treatment at a time at which the chamber wall completely closes the holding chamber.

The moisture content inside the holding chamber 13 can be easily raised, for example before closing the wall 11 and therefore before treating the objects, by spraying the inner face of the wall 11 with water vapor, for example. A raised water vapor portion may make ozone generation more efficient.

In all illustrated embodiments, it is particularly significant to select the distances of the electrodes 16a and 16b and 21 from each other suitably and also to select the voltage suitably, so that gas discharge occurs only inside the holding chamber 13 and not outside the holding chamber 13.

1. An apparatus for treating objects with the help of an electric discharge in a holding chamber for the objects, which chamber is defined by a wall made of dielectric material, at the outer face of which wall at least two electrodes are mounted, characterized in that at least one counter-electrode that is coupled in a capacitive manner to the two outer electrodes is fixed on the inner face of the wall.

2. The apparatus according to claim 1 wherein the apparatus is configured as a transport container and/or as a storage container for the objects.

3. The apparatus according to claim 2 wherein the wall is formed by conventional packaging for the objects, with the exception of the electrodes.

4. The apparatus according to claim 1 wherein the wall is made of plastic, particularly of PE, PA, PVC, PET or the like, or alternatively of a composite material, comprising in particular paper, cardboard, paperboard and plastic.

5. The apparatus according to claim 1 wherein the wall is impermeable to gas.

6. The apparatus according to claim 1 wherein the apparatus is configured as single-use packaging.

7. The apparatus according to claim 1 wherein the apparatus is configured as reusable packaging.

8. The apparatus according to claim 1 wherein the two outer electrodes can be brought into contact with terminals of a power supply.

9. The apparatus according to claim 1 wherein the wall comprises a closable access opening to the holding chamber.

10. The apparatus according to claim 9 wherein the access opening is formed by a door or by a slide fastener.

11. The apparatus according to claim 1 wherein the wall is configured to be flexible either in sections or in its entirety.

12. The apparatus according to claim 1 wherein an inner face and/or an edge of the inner electrode is exposed in the holding chamber.

13. The apparatus according to claim 1 wherein the inner electrode is positioned relative to the outer electrodes and dimensioned such that a transverse projection of the inner electrode on the outer electrodes the outer edge of the inner electrode lies substantially completely inside the outer edge of the outer electrodes.

14. The apparatus according to claim 1 wherein the inner electrode is configured substantially dumbbell-shaped.

15. The apparatus according to claim 14 wherein the inner electrode comprises two heads that are connected to each other.

16. The apparatus according to claim 15 wherein the surface of one head is smaller than the surface of an associated outer electrode opposite it.

17. The apparatus according to claim 15 wherein at least one head of the inner electrode, has a shape with a plurality of areas with directional changes.

18. The apparatus according to claim 1 wherein the inner electrode is substantially symmetrical to a plane of symmetry.

19. The apparatus according to claim 18, characterized in that the two outer electrodes are mounted symmetrically relative to the plane of symmetry.

20. The apparatus according to claim 1 wherein the pressure inside the holding chamber is higher than outside the chamber.

21. The apparatus according to claim 1 wherein a pressure ranging between 50 and 150 hPa, particularly atmospheric pressure, prevails inside the holding chamber during operation of the discharge.

22. The apparatus according to claim 1 wherein the discharge generates ozone and/or UV radiation inside the holding chamber.

23. The apparatus according to claim 1 wherein the holding chamber comprises a gas mixture, particularly an oxygen-containing gas such as air, in addition to the objects to be treated.

24. The apparatus according to claim 1 wherein the water vapor portion in the holding chamber is raised, particularly by supplying water vapor.

25. The apparatus according to claim 1 wherein the inner electrode is attached directly to the wall without leaving an air gap.

26. The apparatus according to claim 1 wherein the outer electrodes are attached directly to the wall without leaving an air gap.

27. The apparatus according to claim 1 wherein the electrodes are made of metal, particularly of silver, gold, stainless steel, aluminum, tin or copper or of an alloy comprising at least one of these metals.

28. The apparatus according to claim 27 wherein at least one electrode is formed by a metal film, which is glued to the wall.

29. The apparatus according to claim 27 wherein at least one electrode is printed, vapor deposited or applied to the wall using a sputter method.

30. The apparatus according to claim 1 wherein at least one electrode is configured as part of a label.

31. The apparatus according to claim 30 wherein the label is attached to the wall.

32. The apparatus claim 1 wherein at least one electrode is made of an optically transparent and electrically conductive material, for example indium tin oxide.

33. The apparatus according to claim 1 wherein an alternating current ranging between 0.5 and 20 kV having a frequency ranging between 100 Hz and 100 MHz, preferably between 1 and 30 kHz, is applied between the two outer electrodes.

34. The apparatus according to claim 1 wherein the discharge is a surface barrier discharge.

35. The apparatus according to claim 1 wherein the discharge is a non-thermal gas discharge, the gas temperature of which is considerably lower than the electron temperature.

36. The apparatus according to claim 1 wherein the thickness of the wall is between 0.05 and 50 mm.

37. The apparatus according to claim 1 wherein an indicator, such as litmus paper, is provided inside the holding chamber, which indicator shows a treatment state of the objects, for example a disinfection level that the objects have reached.
